(19) United States
(12) Patent Application Publication
(10) Pub. No.: US 2024/0423948 A1
(43) Pub. Date: Dec. 26, 2024

(54) 5-HYDROXYMETHYLFURFURAL FOR THE TREATMENT OF MITOCHONDRIAL DYSFUNCTION

(71) Applicant: D-CAP LLC, Caguas, PR (US)

(72) Inventors: Kai DEUSCH, Schlederloh, Icking (DE); Saleh IBRAHIM, Montgelasstr, Munchen (DE)

(73) Assignee: D-CAP LLC, Caguas, PR (US)

(21) Appl. No.: 18/700,452

(22) PCT Filed: Oct. 11, 2021
(86) PCT No.: PCT/EP2021/078081

§ 371 Date: Apr. 11, 2024

(51) Int. Cl. A61K 31/341; A61K 31/10; A61K 31/122; A61K 31/132; A61K 31/165; A61K 31/19; A61K 31/198; A61K 31/201; A61K 31/216; A61K 31/352; A61K 31/4745; A61P 3/00

(52) U.S. Cl. CPC A61K 31/341; A61K 31/10; A61K 31/122; A61K 31/132; A61K 31/165; A61K 31/19; A61K 31/198; A61K 31/201; A61K 31/216; A61K 31/352; A61K 31/4745; A61P 3/00

(57) ABSTRACT

The present invention relates to the use of 5-Hydroxymethylfurfural (5-HMF) for prevention and/or treatment of conditions or diseases, respectively, associated with mitochondrial dysfunction as well as corresponding treatment methods.

The present invention relates to the use of 5-Hydroxymethylfurfural (5-HMF) for prevention and/or treatment of conditions or diseases, respectively, associated with mitochondrial dysfunction as well as corresponding treatment methods.

Mitochondria are cytosolic organelles, and the center of cellular metabolism, producing cellular energy in the form of ATP of which the majority is generated in the oxidative phosphorylation (OXPHOS) machinery (Wallace et al. (2010) Annu. Rev. Pathol. 5, 297-348). They also produce reactive oxygen species (ROS) as a by-product of OXPHOS reaction, which function as signaling molecules (Chandel (2014) BMC Biol. May 27, 12-34). Mitochondrial dysfunction leads to metabolic and chronic inflammatory diseases as well as ageing (Wallace (2005) Annu. Rev. Genet. 39, 359-407). Mitochondria have their own DNA, the mitochondrial DNA (mtDNA) that consists of 13 protein-coding genes as well as 22 transfer RNA and 2 ribosomal RNA genes (Anderson et al. (1981) Nature 9, 290 (5806), 457-465). It thus encodes only a small fraction of the 1500 mitochondrial proteins, the rest of which, are encoded by nuclear genome (nDNA). Mutations in the mtDNA can cause mitochondrial dysfunction, and somatic mutations in mtDNA arise and cumulate over the life (Bratic and Larsson (2013) J. Clin. Invest. 123 (3), 951-957). Therefore, it is not surprising that ageing and age-related disorders (in particular, metabolic disorders, cardiovascular diseases, and neurodegenerative diseases) have been shown to be associated with mtDNA mutations and mitochondrial dysfunction (reviewed in Wallace (2005) Annu. Rev. Genet. 39, 359-407). Thus, mitochondrially directed therapeutic strategies to improve mitochondrial health and function have been proposed to treat many human diseases and promote healthy aging (Webb et al. (2019) Bioorg. Med. Chem. Lett. 299 (11), 1270-1277). For example, calorie restriction is known to be the most robust intervention that increases healthspan and reduces the rate of ageing in a variety of organisms. It stimulates the mitochondrial bioenergetics via a peroxisome proliferation-activated receptor coactivator 1α signaling pathway and is associated by lower ROS production and low oxygen consumption of mitochondria (López-Lluch et al. (2006) Proc. Natl. Acad. Sci. U.S.A. 103 (6), 1768-1773). Antioxidants targeting to mitochondria (e.g., mitoquinol mesylate) are also capable to improve vascular function in healthy older adults by reducing mitochondrial ROS (Rossman et al. (2018) Hypertention 71 (6), 1056-1063). Another example is the chronic moderate inhibition of mitochondrial respiration through cytochrome c oxidase which improves energy homeostasis and metabolic phenotypes such as obesity by reduction of ATP synthesis, which effectively increases mitochondrial bioenergetics and reduces ROS reduction (Tavallaie et al. (2020) Proc. Natl. Acad. Sci. U.S.A. 117 (18), 9840-9850).

Metformin is the most frequently used antidiabetic but has also been implicated in general health improvement and extending lifespan (De Haes et al. (2014) Proc. Natl. Acad. Sci. U.S.A 111 (24), E2501-E2509). Metformin acts on complex I of the respiratory chain (see, e.g. review by Fontaine (2018) From Endocrinol. (Lausanne) 9, 752).

The technical problem underlying the present invention is to provide a novel system for alleviating the consequences of mitochondrial dysfunction.

The solution to the above technical problem is provided by the embodiments of the present invention disclosed in the present description, the claims and the figures.

In particular, the present invention provides the use of 5-HMF in the prevention and/or treatment of conditions and/or diseases associated with mitochondrial dysfunction.

Preferred conditions and/or diseases to which 5-HMF can be applied according to the invention include metabolic disorders, vascular conditions, immunological disorders, cognitive disorders such as neurodegenerative disorders, and malignant disorders. The inventive treatment/prevention of conditions or diseases, respectively, associated with mitochondrial dysfunction also comprises the use of 5-HMF for anti-aging therapy. In particular, many of the specific diseases or conditions, respectively, as outlined before and herein below often occur in a later age of an individual or subject. In that sense “anti-aging” therapy according to the invention comprises a prevention or at least delay of the onset of such conditions and/or diseases.

According to the invention “treatment” means not only that a certain disease or condition is cured. It is also understood according to the invention that “treatment” can mean that the progression of a certain disease stage or stage of a condition, in particular of a pathological condition, is delayed. In other embodiments, “treatment” also comprises that a certain disease or condition does not further progress. Likewise “prevention” also is not confined to a complete circumvention of the specific disease or condition, but can also comprise a limited and/or delayed outbreak of a condition or disease, and can also comprise that a certain, e.g. advanced, stage of a disease or condition is at least delayed or prevented.

Preferred metabolic disorders include type 2 diabetes mellitus, pre-diabetic conditions, metabolic syndrome, alcoholic fatty liver disease, alcoholic steato hepatosis, non-alcoholic fatty liver diseases (NAFLD) and non-alcoholic steato hepatosis (NASH).

Preferred pre-diabetic conditions include insulin resistance and obesity.

Vascular disorders are preferably cardiovascular diseases, cerebral sclerosis, and cerebrovascular diseases.

Cardiovascular diseases in the context of the invention preferably include coronary artery diseases (CAD) such as angina, myocardial infarction (heart attack), heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease and venous thrombosis. Cerebrovascular diseases addressed by the invention preferably include ischemic stroke, embolic stroke, hemorrhagic stroke, transient ischemic attack (TIA), cerebral aneurysm, subarachnoid hemorrhage and cerebrovascular malformation.

Preferred neurodegenerative disorders in the context of the invention include Alzheimer's disease, Parkinson's disease, Huntington's disease, multiple sclerosis, amyotrophic lateral sclerosis, Batten disease, prion disease, progressive bulbar palsy (PBP). pseudobulbar palsy, progressive muscular atrophy (PMA), primary lateral sclerosis (PLS) spinal muscular atrophy (SMA) and monomelic amyotrophy (MMA).

Malignant disorders are preferably selected from tumor and cancer diseases.

More preferably, the malignant disorder is selected from gastrointestinal carcinoid tumors, atypical teratoid tumor, rhabdoid tumor, bronchial tumors, cns embryonal tumors, preferably medulloblastoma, germ cell tumors, extragonadal germ cell tumor, extracranial germ cell tumors, gastrointestinal carcinoid tumor, gastrointestinal stromal tumors (GIST), intraocular melanoma, tracheobronchial tumor, melanoma, pancreatic neuroendocrine tumors, pituitary tumor, acute lymphoblastic leukemia (ALL), acute myeloid leukemia (AML), adrenocortical carcinoma, Kaposi sarcoma, aids-related lymphoma, primary CNS lymphoma, anal cancer, appendix cancer, astrocytomas, brain cancer, basal cell carcinoma of the skin, bile duct cancer, bladder cancer, bone cancer, Ewing sarcoma, osteosarcoma, malignant fibrous histiocytoma, female breast cancer, lung cancer, Burkitt lymphoma, primary CNS lymphoma, cervical cancer, cholangiocarcinoma, chordoma, chronic lymphocytic leukemia (CLL), chronic myelogenous leukemia (CML), chronic myeloproliferative neoplasms, colorectal cancer, craniopharyngioma, cutaneous T-cell lymphoma, ductal carcinoma in situ (DCIS), endometrial cancer, ependymoma, esophageal cancer, esthesioneuroblastoma, eye cancer, retinoblastoma, fallopian tube cancer, fibrous histiocytoma of bone, gallbladder cancer, gastric cancer, testicular cancer, gestational trophoblastic disease, hairy cell leukemia, head and neck cancer, liver cancer, histiocytosis, Langerhans cell Hodgkin lymphoma, hypopharyngeal cancer, kidney cancer, Langerhans cell histiocytosis, laryngeal cancer, leukemia, lip and oral cavity cancer, lung cancer such as small cell lung cancer, small cell lung cancer and pleuropulmonary blastoma, lymphoma, male breast cancer, malignant fibrous histiocytoma of bone, Merkel cell carcinoma, malignant mesothelioma, metastatic cancer, metastatic squamous neck cancer, midline tract carcinoma, mouth cancer, multiple endocrine neoplasia syndromes, multiple myeloma/plasma cell neoplasms, mycosis fungoides, myelodysplastic syndromes, myelodysplastic/myeloproliferative neoplasms, chronic myelogenous leukemia, chronic (CML), acute myeloid leukemia (AML), chronic myeloproliferative neoplasms, nasal cavity and paranasal sinus cancer, nasopharyngeal cancer, neuroblastoma, non-Hodgkin lymphoma, non-small cell lung cancer, oral cancer, lip and oral cavity cancer, oropharyngeal cancer, undifferentiated pleomorphic sarcoma of bone treatment, ovarian cancer. pancreatic cancer, papillomatosis, paraganglioma, paranasal sinus and nasal cavity cancer, parathyroid cancer, penile cancer, pharyngeal cancer, pheochromocytoma, plasma cell neoplasm/multiple myeloma, pleuropulmonary blastoma, primary central nervous system (CNS) lymphoma, primary peritoneal cancer, prostate cancer, rectal cancer, recurrent cancer, retinoblastoma, salivary gland cancer, sarcoma, childhood vascular tumors, soft tissue sarcoma, uterine sarcoma, Sezary syndrome, skin cancer, small cell lung cancer, small intestine cancer, soft tissue sarcoma, squamous neck cancer with occult primary, stomach (gastric) cancer, testicular cancer, throat cancer, nasopharyngeal cancer, oropharyngeal cancer, hypopharyngeal cancer, thymoma, thymic carcinoma, thyroid cancer, tracheobronchial tumors, transitional cell cancer of the renal pelvis and ureter, transitional cell cancer, urethral cancer, uterine cancer, endometrial uterine sarcoma, vaginal cancer, vascular tumors, vulvar cancer and Wilms tumor.

Preferred immunological disorders in the context of the invention include allergies, asthma, autoimmune diseases, autoinflammatory syndromes and immunological deficiency syndromes.

According to the invention, 5-HMF may be used alone as a single therapy.

In other embodiments of the invention 5-HMF is preferably used in combination with at least one further active agent, preferably at least one further agent for modulation of mitochondrial function.

In embodiments of the invention, the selected one or more further agent(s) is/are preferably selected such that it/they complement and/or support the function of 5-HMF in a certain use of same as disclosed herein, more preferably its function for preventing and/or treating one or more selected disease states or conditions (or group of diseases or conditions). Particularly preferred combinations of the invention and uses thereof for certain diseases/conditions are further outlined below.

Preferred agents for combinatorial use of 5-HMF include natural substance, i.e. substances that occur in nature such as from terrestrial and/or arial and/or marine sources such as from animal and/or plants. Such natural substances may be used in purified form and/or in form of one or more extracts from an original source of the natural active agent. In embodiments of the invention sources of the natural substance include extracts of a complete source such as a plant, or, in other embodiments, extracts of a certain part of the natural source such as an extract of a part of a plant, preferably a part of a plant known to comprise the natural substance, more preferably a part where the natural substance is enriched in comparison to other parts of the plant, e.g. an extract from a root, from leaves, fruits and/or from the stem of a plant. In other embodiments, the natural substances may be derived from one or more parts of animals where the active substance is present, preferably a part where the active substance is enriched in comparison to other parts of the animal, such as bone, body fluids such as blood, plasma, interstitial fluid, sweat, tears, saliva, urine and liquor, organs such as heart, kidney, liver, brain, gut, stomach skin, eyes etc. Particularly preferred natural substances as additional agents for use in the invention include apigenin, quinine, pyrrolochinoline quinone (PQQ), sulphoraphane, nonivamide, capsaicin, dehydrocapsiate, hyperforin allicin, DHC, ascorbic acid, spermidine, oleate, propionate, L-lysine, D-glucose, Utrolithin A, selenium, zinc, N-acylcysteine, Nacystelyn and N-acylcysteine amide.

It is to be understood according to the invention that the term “ascorbic acid” includes the free acid as well as its salts, preferably alkaline metal salts such as sodium ascorbate and/or potassium ascorbate, or alkaline earth metal salts such as calcium ascorbate and/or manganese ascorbate.

According to the invention, the term “oleate” refers, on the one hand, to the anion of oleic acid which can be present, in particular having regard to compositions as disclosed herein, in the form of oleic acid salts, preferably alkaline metal salts such as sodium oleate and/or potassium oleate, or alkaline earth metal salts such as calcium oleate and/or manganese oleate. On the other hand, the term “oleate” as used herein refers in certain embodiments to esters of oleic esters such as lower alkyl esters, preferably C₁₋₄-alkyl esters of oleic esters, more preferably methyl oleate, ethyl oleate, propyl oleate, butyl oleate or a mixture of two or more thereof. Other preferred esters of oleic acid for use in the invention include glyceryl and cetyl esters of oleic acid. In certain embodiments of the invention, the term “oleate” also refers to the free oleic acid. It is also included in the invention that mixtures including two or more of the above-described forms, namely free oleic acid, oleic acid salts and/or oleic acid esters can be used.

According to the invention, the term “propionate” refers, on the one hand, to the anion of propionic acid which can be present, in particular having regard to compositions as disclosed herein, in the form of propionic acid salts, preferably alkaline metal salts such as sodium propionate and/or potassium propionate, or alkaline earth metal salts such as calcium propionate and/or manganese propionate.

On the other hand, the term “propionate” as used herein refers in certain embodiments to esters of oleic esters such as lower alkyl esters, preferably C₁₋₄-alkyl esters of propionic acid esters, more preferably methyl propionate, ethyl propionate, propyl propionate, butyl propionate or a mixture of two or more thereof. Other preferred esters of propionic acid for use in the invention include glyceryl and cetyl esters of propionic acid. It is also included in the invention that mixtures including two or more of the above-described forms, namely propionic acid salts and/or propionic acid esters can be used.

According to the invention, the term “selenium” may comprise elementary selenium, but in preferred embodiments of the invention it preferably refers to, and is preferably used in the invention as, a selenium salt, more preferably as selenit salt, preferably alkaline metal salts such as sodium selenit and/or potassium selenite, or alkaline earth metal salts such as calcium selenit and/or manganese selenit.

According to the invention, the term “zinc” may comprise elementary selenium, but in preferred embodiments of the invention it preferably refers to, and is preferably used in the invention as, a zinc salt such as zinc sulfate, zinc acetate and zinc bis-glycinate.

In other embodiments of the invention, the at least one further agent may be selected from non-natural substances. In preferred embodiments non-natural substances or compounds, respectively, for modulation of mitochondrial function can be selected from metformin, statins such as preferably one or more selected from atorvastatin, fluvastatin, lovastatin, pitavastatin, pravastatin, rosuvastatin calcium and simvastatin, and thiazolidinediones such as preferably one or more selected from rosiglitazone, troglitazone, and pioglitazone.

It is to be understood that combinatorial applications of 5-HMF together with at least one further active agent as outlined herein can be practiced by administrating 5-HMF simultaneously or sequentially together with the at least one further active agent. Furthermore, in certain embodiments of the invention, simultaneous administration of 5-HMF and the at least one agent preferably comprises administration of 5-HMF and the at least one further agent in a single composition, preferably a single pharmaceutical composition. In other embodiments, e.g. in situations where 5-HMF and a further active agent (or further active agents) are chemically and/or pharmacologically not well combinable, 5-HMF and the further active agent(s) may be administered in different compositions, preferably different pharmaceutical compositions. In this context, it is to be understood that “simultaneous” administration also includes separated administration of an individual compositions containing 5-HMF and of an individual composition containing the at least one further agent in immediate sequential administration, and such administration is considered as “essential simultaneous administration”. The specific route of administration of 5-HMF according to the invention and of that of the one or more further active agents, depends on various factors such as type of agent, disease or condition, sex and age of the individual or subject, preferably a human, to be treated. Systemic administration is generally preferred, with oral administration being the particularly preferred route of administration. In other embodiments, intravenous administration may equally or preferably be chosen.

5-HMF and other active agents for use in the invention are preferably administered in the form of pharmaceutical compositions containing one or more active agents as outlined herein and at least one pharmaceutically acceptable vehicle, excipient and/or diluent. In other embodiments, 5-HMF and/or other active agents for use in the invention may also be formulated into nutraceutical compositions or food supplement compositions. In still other embodiments, 5-HMF and/or other active agent(s) for use in the invention may be mixed directly with food.

5-HMF and/or the further active agent(s) may be administered in one or more unit dosages, preferably once or more daily, such as twice or thrice daily. Preferably, a total dosage or unit dosage leads to a suitable pharmacological plasma concentration of the active agent which is considered according to the invention as an “effective dose” or “effective amount”, respectively, of the active agent(s).

Preferably, 5-HMF and/or the at least one further active agent is administered in a dose leading to a plasma concentration of from about 0.01 μM to 10 mM, preferably from about 0.02 μM to about 1 mM, more preferably from about 0.05 to about 0.5mM, most preferred from about 0.05 μM to about 0.2 μM, particularly preferred about 0.1 μM.

The subject or individual, respectively, to be treated according to the invention is preferably a mammal, more preferably a human.

In preferred embodiments of the invention, 5-HMF is used in combination with ascorbic acid and/or Spermidine and/or Urolithin A. In one preferred embodiment, 5-HMF is used in combination with ascorbic acid and Spermidine. In a further preferred embodiment 5-HMF is used in combination with ascorbic acid, Spermidine and Urolithin A. Such inventive combinations of 5-HMF are particularly useful for anti-aging therapy.

In other preferred embodiments of the invention 5-HMF is used in combination with dehydrocapsiate and/or oleic acid and/or propionate and/or L-lysine and/or Nonivamide and/or capsaicin. In one embodiment, 5-HMF is preferably used in combination with dehydrocapsiate, oleic acid, propionate an L-lysine. In other preferred embodiments, 5-HMF is used in combination with oleic acid, propionate, L-lysine and Nonivamide. In still other preferred embodiments 5-HMF is preferably used in combination with oleic acid, propionate, L-lysine and capsaicin. Such inventive combinations of 5-HMF are particularly useful for prevention and/or treatment of metabolic disorders.

In further preferred embodiments of the invention 5-HMF is used in combination with Apigenin and/or hyperforin and/or PQQ and/or Urolithin A. In one embodiment, 5-HMF is preferably used in combination with Apigenin, hyperforin and PQQ. In other embodiments 5-HMF is preferably used in combination with Apigenin, hyperforin and Urolithin. A. In still other preferred embodiments 5-HMF is used in combination with Apigenin, hyperforin, PQQ and Urolithin A. Such inventive combinations of 5-HMF are particularly useful for prevention and/or treatment of cognitive disorders, preferably neurodegenerative diseases.

In further preferred embodiments of the invention 5-HMF is used in combination with sulphoraphane and/or allicin and/or PQQ and/or propionate and/or Urolithin A. In a preferred embodiment, 5-HMF is used in combination with sulphoraphane, allicin and propionate. In an alternative embodiment 5-HMF is preferably used in combination with sulphoraphane, PQQ and propionate. In still a further preferred embodiment 5-HMF is used in combination with sulphoraphane, PQQ, propionate and Urolithin A. in yet a further preferred embodiment 5-HMF is used in combination with sulphoraphane, propionate and Urolithin A. Such inventive combinations of 5-HMF are particularly useful for prevention and/or treatment of immunological diseases or disorders.

In a further aspect of the invention, there is provided a composition, preferably in form of a pharmaceutical composition, comprising at least two active agents selected from the group consisting of sulphoraphane, allicin, PQQ, propionate and Urolithin A. In a preferred embodiment, the composition contains sulphoraphane, allicin and propionate. In an alternative embodiment, the composition contains sulphoraphane, PQQ and propionate. In still a further preferred embodiment the composition contains sulphoraphane, PQQ, propionate and Urolithin A. in yet a further preferred embodiment, the composition contains sulphoraphane, propionate and Urolithin A. Such inventive compositions are particularly useful for prevention and/or treatment of immunological diseases or disorders.

The invention is furthermore directed to a composition comprising 5-HMF and at least one further active agent, preferably at least one further agent for modulation of mitochondrial function. Preferably, the composition of the invention is a pharmaceutical composition comprising 5-HMF and the at least one active agent, preferably at least one further agent for modulation of mitochondrial function, together with at least one pharmaceutical carrier, excipient and/or diluent. In other embodiments, the composition of the invention is in the form of a nutraceutical composition comprising 5-HMF and the at least one active agent, preferably at least one further agent for modulation of mitochondrial function, together with at least one neutraceutically acceptable carrier, excipient and/or diluent. Thus, the composition of the invention may also be in form of a nutritional supplement.

Preferred pharmaceutical and nutraceutical, respectively, compositions of the invention are in the form of oral dosages forms such as tablets, capsules including minicapsules and microcapsules, lozenges, syrups, solutions, drinks, powders, pastes, thin films and the like.

Preferred combinations of 5-HMF with at least one active agent for the inventive composition have already been elaborated herein above with respect to the inventive use of 5-HMF.

The invention also relates to a method of treatment and/or prevention of conditions and/or diseases associated with mitochondrial dysfunction comprising the step of administering an effective amount of 5-HMF to a subject in need thereof. As outlined before, 5-HMF can be combined in the inventive treatment method with one or more active agents, preferably one or more of those as outlined before. The same applies with respect to administration routes, dosages, and administration regimes.

The Figures show:

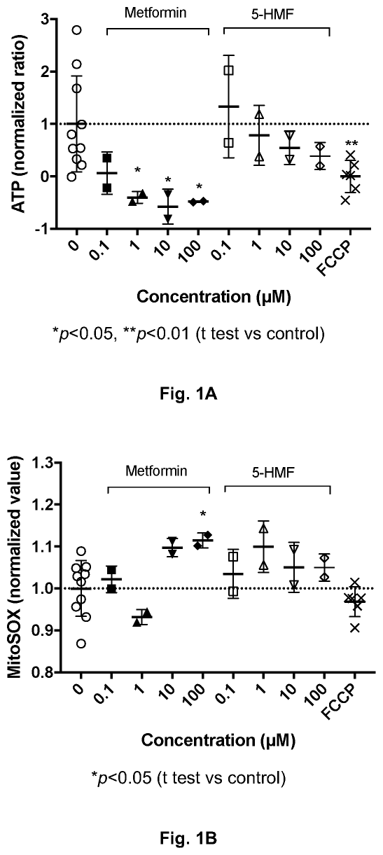

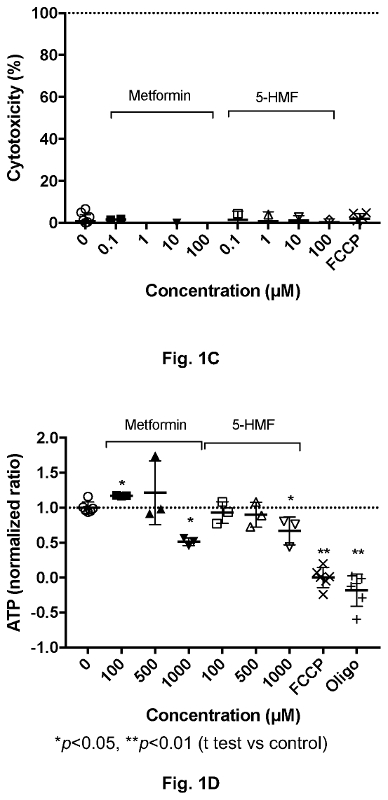

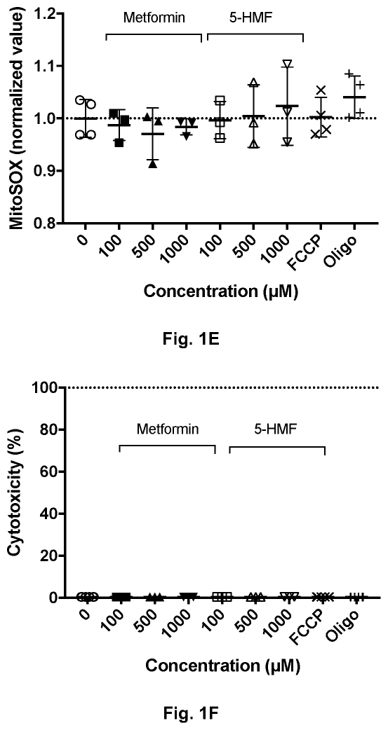

FIG. 1: Dose dependency effect of metformin and 5-HMF on mitochondrial function. A-C. 0.1-100 μM, D-F. 100-100 μM concentration. A, D. ATP levels; B, E. MitoSOX; C, F. LDH (cytotoxicity) assay. 5-HMF effect on mitochondrial function (ATP levels, mitochondrial ROS levels) is comparable to that of metformin.

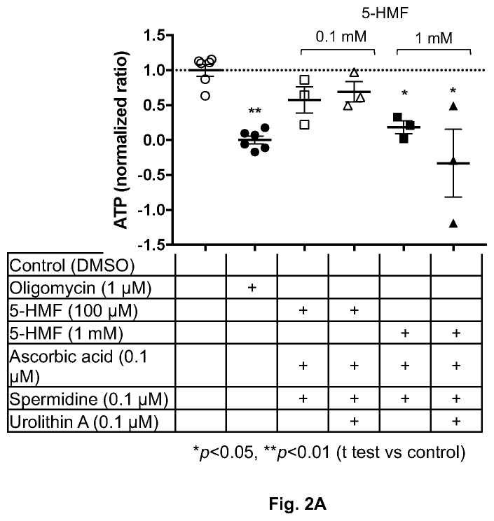

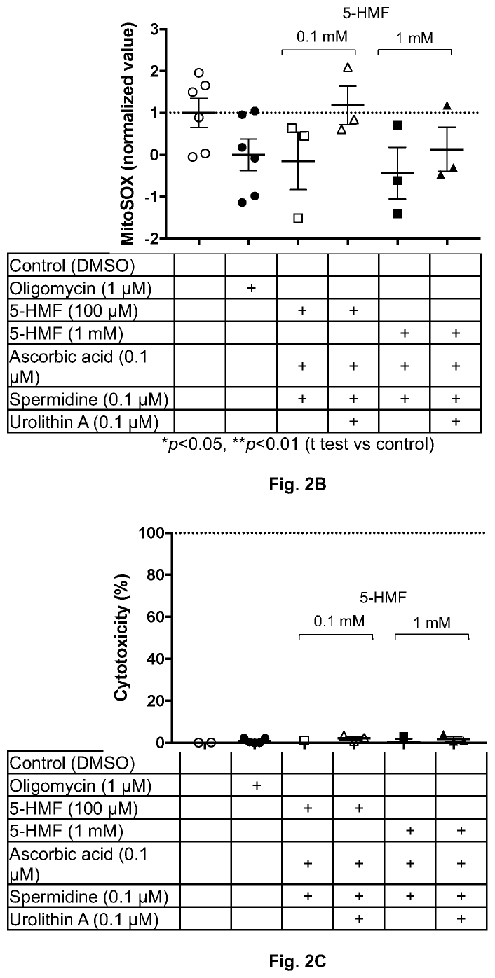

FIG. 2: Results of experiments showing the effect of preferred inventive combinations of 5-HMF and further agents for use in anti-aging. A. ATP levels. High dose 5-HMF (1 mM) combination showed stronger ATP reduction. B. MitoSOX levels. Due to the data variation, no statistically significance was observed (even oligomycin, which is positive control), suggesting no major changes in mitochondrial ROS production. C. LDH levels (cytotoxicity). All compound combinations did not induce cell deaths. Cells treated with 0.1% Triton-X are positive control (100%), and control (DMSO) cells are negative control (0%).

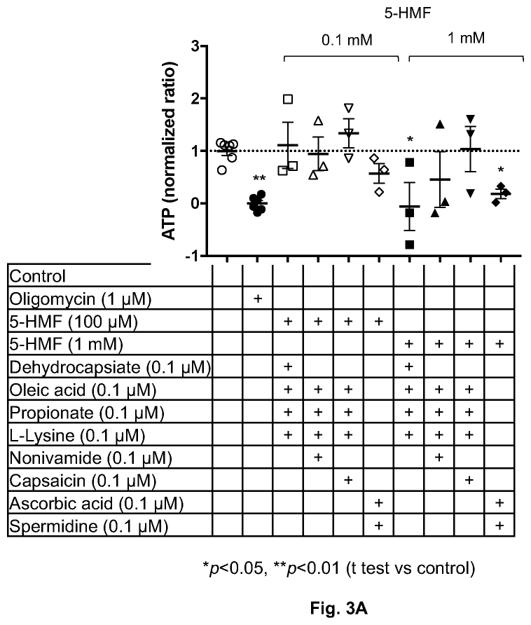

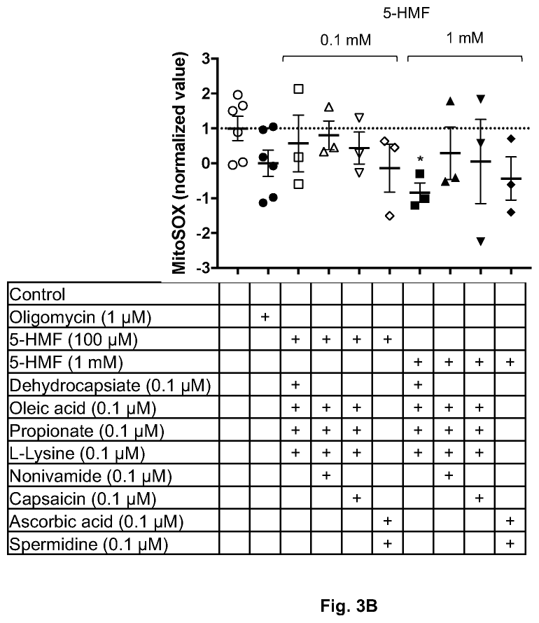

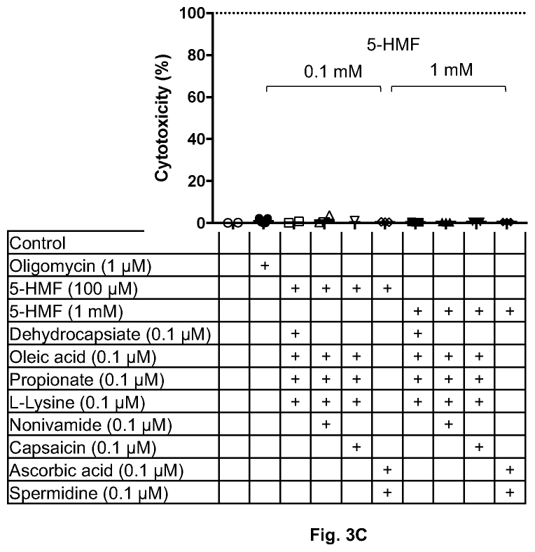

FIG. 3: Results of experiments showing the effect of preferred inventive combinations of 5-HMF and further agents for use in therapy and/or prevention of metabolic conditions. A. ATP levels. High dose 5-HMF (1 mM) combination showed stronger ATP reduction. B. MitoSOX levels. Due to the data variation, no statistically significance was observed (even oligomycin, which served as positive control), suggesting no major changes in mitochondrial ROS production. C. LDH levels (cytotoxicity). All compound combinations did not induce cell deaths. Cells treated with 0.1% Triton-X are positive control (100%), and control (DMSO) cells are negative control (0%).

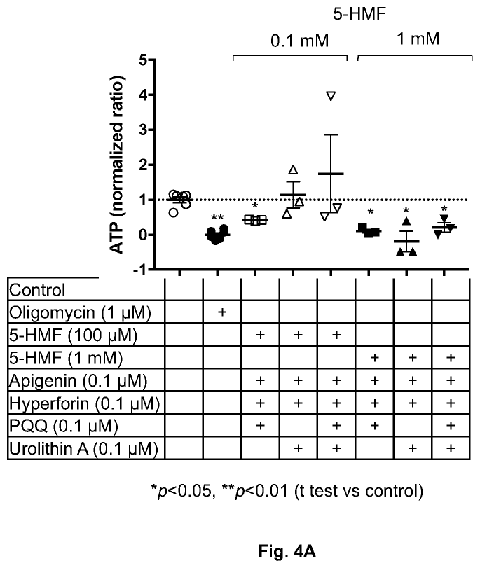

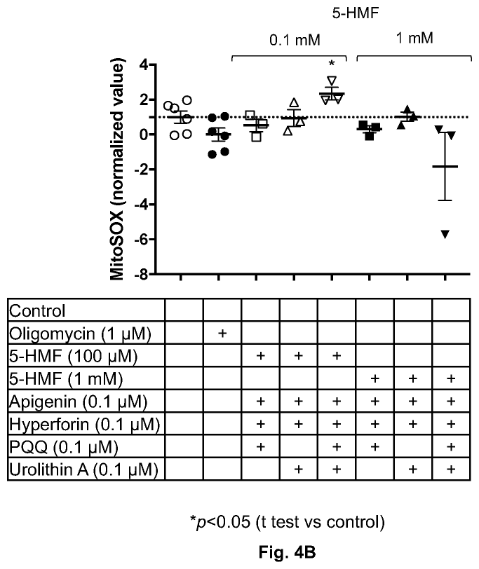

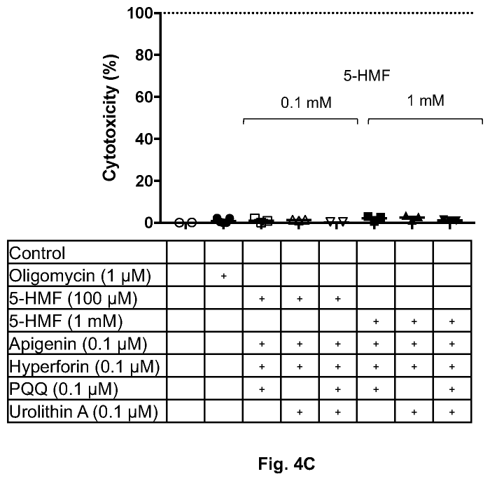

FIG. 4: Results of experiments showing the effect of preferred inventive combinations of 5-HMF and further agents for use in therapy and/or prevention of cognitive disorders. A. ATP levels. High dose 5-HMF (1 mM) combination showed stronger ATP reduction. B. MitoSOX levels. Higher dose 5-HMF (1 mM) did not change mitochondrial ROS levels. C. LDH levels (cytotoxicity). All compound combinations did not induce cell deaths. Cells treated with 0.1% Triton-X are positive control (100%) and control (DMSO) cells are negative control (0%).

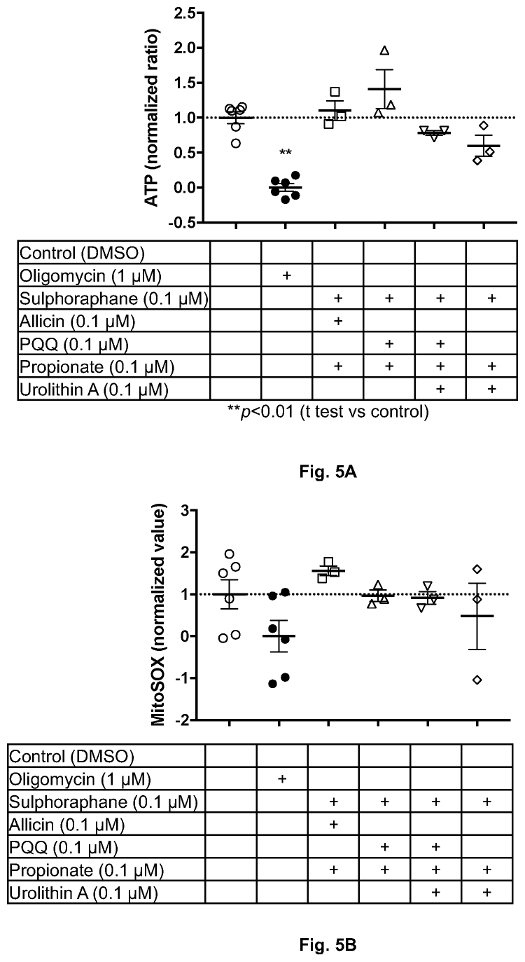

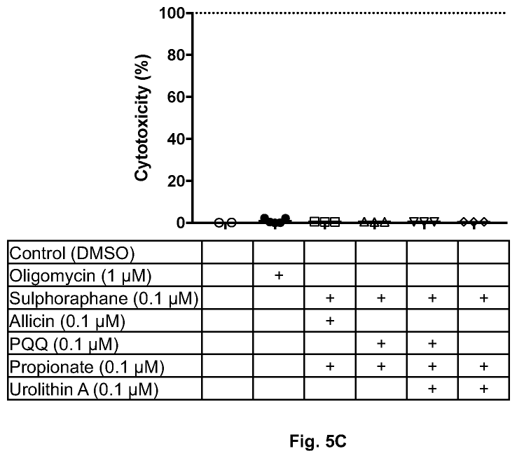

FIG. 5: Results of experiments showing the effect of preferred inventive combinations of natural substances for use in therapy and prevention of immune disorders.

### EXAMPLES

### Example 1: Methods

Compound preparation: Compounds were dissolved in DMSO, EtOH, or water, depending on the solubility of the compound. 10% DMSO in PBS was used to prepare the working solution. The final concentration of DMSO did not exceed 0.1%.

Cell culture: The Hepa1-6 cell line (#CRL-1830, ATCC) was cultured in DMEM culture medium containing 1 g/L glucose, 10% fetal bovine serum, 2 mM glutamine, 100 U/mL Penicillin and 100 μg/mL Streptomycin and 30 μM oleic acid. Cells were seeded at a density of 20 000 cells per well in 96 well plates and incubated for overnight to allow the cells to attach at 37° C. with 5% CO₂. Cells were then supplemented with the test substances at indicated concentrations and incubated for indicated time periods (24 h).

For all assays the test substances were diluted in 10% DMSO/PBS further diluted with culture medium and added to the cells at the indicated concentrations with a final DMSO concentration of 0.01%. Vehicle controls were treated with 0.01% DMSO.

ATP content: Cells were treated for 24 h with the test substances at the indicated concentrations. The cellular ATP content was assessed using the CellTiter-Glo Assay (#G7571, Promega) as per the manufacturer's instructions. Luminescence was detected in a plate reader spectrophotometer. The results were normalized to the luminescence intensity measured in vehicle treated cells and positive controls treated with 0.1% Triton-X, as well as 10 μM FCCP and 1 μM oligomycin. Flat, white bottom plate.

Mitochondrial reactive oxygen species (ROS); Cells were treated for 24 h with the test substances at the indicated concentration, as well as positive (10 μM FCCP and 1 μM oligomycin) and negative control. Mitochondrial ROS was detected using MitoSOX™ Red mitochondrial superoxide indicator (#M36008, Invitrogen/Thermo Fisher Scientific). After 24 h treatment of the substances, cells were washed with 100 μl assay buffer (DMEM without phenol red with 4 mM L-glutamine, 4.5 g/L glucose and 1 mM Na-Pyruvate, pH7.4) three times, and 100 μl of 1.5 μM MitoSOX Red (5 mM MitoSOX™ Red stock solution prepared as indicated by the manufacturer's instruction diluted in 1:2 500 in assay buffer) was added per well, followed by incubation for 30min at 37° C. with 5% CO₂. After the incubation cells were washed with 100 μl assay buffer and 100 μl assay buffer was added per well. Fluorescence was measured at excitation 510 nm and emission 580 nm. Use flat, black bottom plates were used.

Cytotoxicity: Cytotoxicity was assessed using the CyQuant™ LDH Cytotoxicity Assay Kit (#C20300/C20301, Thermo Fisher). After cells were cultured with the test substances for 24 h, 50 μL of the cell culture medium were transferred to a new 96 well plate and the cytotoxicity assay was performed as per the manufacturer's instructions. Absorbance was measured at 490 nm and background at 680 nm. The signal intensity of untreated cells was considered as the background signal of spontaneous LDH release and subtracted from all measurements. Cytotoxicity was expressed as the percentage of maximum signal intensity observed in freshly lysed cells. Flat, clear plate. 10 μM FCCP, 1 μM oligomycin, 0.1% Triton X as positive control.

Statistical analysis: Statistical analysis was performed using GraphPad Prism v6.07 (GraphPad Software), and statistical tests include one-way ANOVA, multiple t test were used.

### Example 2: 5-HMF is an Excellent Replacement of Metformin

Impact of Metformin and 5-HMF on mitochondrial function, i.e., ATP levels (FIG. 1A and 1D) and MitoSOX (FIG. 1B and 1E) and cytotoxicity (FIG. 1C and 1F) was evaluated on murine hepatocyte cell line at different concentrations (a lower dose range: FIG. 1A-C; a higher dose range: FIG. 1D-F). In general, both of Metformin and 5-HMF treatments showed ATP reduction (FIG. 1A and 1D) and a trend of slight increase of MitoSOX levels (FIG. 1B and 1E) without cytotoxic effects (FIG. 1C and 1F). Remarkably, at a lower dose range, which is at physiological doses of Metformin, the effect of 5-HMF on mitochondrial function were milder than that of Metformin (FIG. 1A and 1B, respectively).

### Example 3:5-HMF Combinations Useful in the Treatment of Age-Related Disorders

Effect of treatment with different mixtures of 3 natural compounds (ascorbic acids, spermidine, and Urolithin A; 2 types of combination; each compound at 0.1 μM concentration) and 5-HMF (100 μM or 1 mM) on mitochondrial function (ATP and mitochondrial ROS levels) in murine hepatocytes were evaluated. ATP levels were significantly reduced when cells were treated with 2 types of combinations with 1 mM dose of 5-HMF than those with 100 μM 5-HMF (FIG. 2A) without inducing cell death (FIG. 2C). Mitochondrial ROS (MitoSOX) levels were not largely affected by the treatment (FIG. 2B).

### Example 4: 5-HMF Combinations Useful in the Treatment of Metabolic Conditions

Effect of treatment with different mixtures of 8 natural compounds (dehydrocapsiate, oleic acid, propionate, L-lysine, nonivamide, capsaicin, ascorbic acid, and spermidine; 4 types of combination; each compound at 0.1 μM concentration) together with 5-HMF (100 μM or 1 mM) on mitochondrial function (ATP and mitochondrial ROS levels) in murine hepatocytes were evaluated. Both ATP and MitoSOX levels were significantly reduced when cells were treated with a combination of dehydrocapsiate, oleic acid, propionate and L-lysine with 1 mM 5-HMF, while those effect were not prominent when treated with the combination with 100 μM 5-HMF (FIG. 3A and 3B). All tested compound combinations did not induce cell death (FIG. 3C).

### Example 5:5-HMF Combinations Useful in the Treatment of Cognitive Disorders

Effect of treatment with different mixtures of 4 natural compounds (apigenin, hyperforin, PQQ, and Urolithin A; 3 types of combination; each compound at 0.1 μM) together with 5-HMF (100 μM or 1 mM) on mitochondrial function (ATP and mitochondrial ROS levels) in murine hepatocytes were evaluated. ATP levels were significantly reduced when cells were treated with all 3 types of combinations with 1mM 5-HMF, but not with 100 μM 5-HMF (FIG. 4A). The levels of MitoSOX were unchanged when treated with 2 combinations (apigenin+hyperforin+PQQ, and apigenin+hyperforin+Urolithin A) regardless the concentration of 5-HMF. All 4natural compound combinations showed significantly increased MitoSOX levels when treated with 100 μM 5-HMF, while those with 1 mM 5-HMF showed a trend of reduction (FIG. 4B). All of these combination treatments did not induce cell death (FIG. 4C).

### Example 6: Combinations of Natural Substances Useful in the Treatment of Immune Disorders

Effect of treatment with different mixture of 4 natural compounds (allicin, PQQ, propionate, Urolithin A; 4 types of combination; each compound at 0.1 μM) on mitochondrial function (ATP and mitochondrial ROS levels) in murine hepatocytes were evaluated. Two combinations with Urolithin A showed mild reduction of ATP levels (FIG. 5A). All four treatment combinations did not cause major changes in MitoSOX levels (FIG. 5B). All of these combination treatments did not induce cell death (FIG. 5C).

1. 5-Hydroxymethylfurfural (5-HMF) for use in the prevention and/or treatment of conditions and/or diseases associated with mitochondrial dysfunction.

2. 5-HMF for use of claim 1 wherein the condition and/or diseases are selected form the group consisting of metabolic disorders, vascular conditions, immunological disorders, neurodegenerative disorders and malignant disorders.

3. 5-HMF for use of claim 1 or 2 wherein 5-HMF is used for anti-aging therapy.

4. 5-HMF for use of claim 2 wherein the metabolic disorder is selected from the group consisting of type 2 diabetes mellitus, pre-diabetic conditions, metabolic syndrome, alcoholic fatty liver disease, alcoholic steato hepatosis, non-alcoholic fatty liver diseases (NAFLD) and non-alcoholic steato hepatosis (NASH).

5. 5-HMF for use of claim 4 wherein the pre-diabetic condition is selected from the group consisting of insulin resistance and obesity.

6. 5-HMF for use of claim 2 wherein the vascular disorder is selected from the group consisting of cardiovascular diseases, cerebral sclerosis, and cerebrovascular diseases.

7. 5-HMF for use of claim 6 wherein the cardiovascular disease is selected from the group consisting of coronary artery diseases (CAD) such as angina, myocardial infarction (heart attack), heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease and venous thrombosis.

8. 5-HMF for use of claim 6 wherein the cerebrovascular diseases is selected from the group consisting of ischemic stroke, embolic stroke, hemorrhagic stroke, transient ischemic attack (TIA), cerebral aneurysm, subarachnoid hemorrhage and cerebrovascular malformation.

9. 5-HMF for use of claim 2 wherein the neurodegenerative disorder is selected from the group consisting of Alzheimer's disease, Parkinson's disease, Huntington's disease, multiple sclerosis, amyotrophic lateral sclerosis, Batten disease, prion disease, progressive bulbar palsy (PBP), pseudobulbar palsy. progressive muscular atrophy (PMA), primary lateral sclerosis (PLS) spinal muscular atrophy (SMA) and monomelic amyotrophy (MMA).

10. 5-HMF for use of claim 2 wherein the malignant disorder is selected from tumor and cancer diseases.

11. 5-HMF for use of claim 10 wherein the disease is selected from the group consisting of gastrointestinal carcinoid tumors, atypical teratoid tumor, rhabdoid tumor, bronchial tumors, cns embryonal tumors, preferably medulloblastoma, germ cell tumors, extragonadal germ cell tumor, extracranial germ cell tumors, gastrointestinal carcinoid tumor, gastrointestinal stromal tumors (GIST), intraocular melanoma, tracheobronchial tumor, melanoma, pancreatic neuroendocrine tumors, pituitary tumor, acute lymphoblastic leukemia (ALL), acute myeloid leukemia (AML), adrenocortical carcinoma, Kaposi sarcoma, aids-related lymphoma, primary CNS lymphoma, anal cancer, appendix cancer, astrocytomas, brain cancer, basal cell carcinoma of the skin, bile duct cancer, bladder cancer, bone cancer, Ewing sarcoma, osteosarcoma, malignant fibrous histiocytoma, female breast cancer, lung cancer, Burkitt lymphoma, primary CNS lymphoma, cervical cancer, cholangiocarcinoma, chordoma, chronic lymphocytic leukemia (CLL), chronic myelogenous leukemia (CML), chronic myeloproliferative neoplasms, colorectal cancer, craniopharyngioma, cutaneous T-cell lymphoma, ductal carcinoma in situ (DCIS), endometrial cancer, ependymoma, esophageal cancer, esthesioneuroblastoma, eye cancer, retinoblastoma, fallopian tube cancer, fibrous histiocytoma of bone, gallbladder cancer, gastric cancer, testicular cancer, gestational trophoblastic disease, hairy cell leukemia, head and neck cancer, liver cancer, histiocytosis, Langerhans cell Hodgkin lymphoma, hypopharyngeal cancer, kidney cancer, Langerhans cell histiocytosis, laryngeal cancer, leukemia, lip and oral cavity cancer, lung cancer such as small cell lung cancer, small cell lung cancer and pleuropulmonary blastoma, lymphoma, male breast cancer, malignant fibrous histiocytoma of bone, Merkel cell carcinoma, malignant mesothelioma, metastatic cancer, metastatic squamous neck cancer, midline tract carcinoma, mouth cancer, multiple endocrine neoplasia syndromes, multiple myeloma/plasma cell neoplasms, mycosis fungoides, myelodysplastic syndromes, myelodysplastic/myeloproliferative neoplasms, chronic myelogenous leukemia, chronic (CML), acute myeloid leukemia (AML), chronic myeloproliferative neoplasms, nasal cavity and paranasal sinus cancer, nasopharyngeal cancer, neuroblastoma, non-Hodgkin lymphoma, non-small cell lung cancer, oral cancer, lip and oral cavity cancer, oropharyngeal cancer, undifferentiated pleomorphic sarcoma of bone treatment, ovarian cancer. pancreatic cancer, papillomatosis, paraganglioma, paranasal sinus and nasal cavity cancer, parathyroid cancer, penile cancer, pharyngeal cancer, pheochromocytoma, plasma cell neoplasm/multiple myeloma, pleuropulmonary blastoma, primary central nervous system (CNS) lymphoma, primary peritoneal cancer, prostate cancer, rectal cancer, recurrent cancer, retinoblastoma, salivary gland cancer, sarcoma, childhood vascular tumors, soft tissue sarcoma, uterine sarcoma, Sezary syndrome, skin cancer, small cell lung cancer, small intestine cancer, soft tissue sarcoma, squamous neck cancer with occult primary, stomach (gastric) cancer, testicular cancer, throat cancer, nasopharyngeal cancer, oropharyngeal cancer, hypopharyngeal cancer, thymoma, thymic carcinoma, thyroid cancer, tracheobronchial tumors, transitional cell cancer of the renal pelvis and ureter, transitional cell cancer, urethral cancer, uterine cancer, endometrial uterine sarcoma, vaginal cancer, vascular tumors, vulvar cancer and Wilms tumor.

12. 5-HMF for use of claim 2 wherein the immunological disorder is selected from allergies, asthma, autoimmune diseases, autoinflammatory syndromes and immunological deficiency syndromes.

13. 5-HMF for use according to any one of the preceding claims wherein 5-HMF is used in combination with at least one further active agent, preferably a further agent for modulation of mitochondrial function.

14. 5-HMF for use of claim 13 wherein the at least one agent is selected from the group consisting of one or more natural substances and one or more non-natural substances.

15. 5-HMF for use of claim 14 wherein the natural substance is selected from the group consisting of apigenin, quinine, PQQ, sulphoraphane, nonivamide, capsaicin, dehydrocapsiate, hyperforin allicin, DHC, ascorbic acid, spermidine, oleate, propionate, L-lysine, D-glucose, Utrolithin A, selenium, zinc, N-acylcysteine, Nacystelyn and N-acylcysteine amide.

16. 5-HMF for use of claim 14 wherein the non-natural substance is selected from metformin, statins and thiazolidinones.

17. 5-HMF for use of claim 15 wherein the statin is selected from the group consisting of one or more of atorvastatin, fluvastatin, lovastatin, pitavastatin, pravastatin, rosuvastatin calcium and simvastatin.

18. 5-HMF for use of claim 16 wherein the thiazolidinone is selected from the group consisting of one or more of rosiglitazone, troglitazone, and pioglitazone.

19. 5-HMF for use of claim 15 wherein 5-HMF is used in combination with ascorbic acid and/or Spermidine and/or Urolithin A.

20. 5-HMF for use of claim 19 wherein 5-HMF is used in combination with ascorbic acid and Spermidine.

21. 5-HMF for use of claim 19 wherein 5-HMF is used in combination with ascorbic acid, Spermidine and Urolithin A.

22. 5-HMF for use of claim 15 wherein 5-HMF is used in combination with dehydrocapsiate and/or oleic acid and/or propionate and/or L-lysine and/or Nonivamide and/or capsaicin.

23. 5-HMF for use of claim 22 wherein 5-HMF is used in combination with dehydrocapsiate, oleic acid, propionate and L-lysine.

24. 5-HMF for use of claim 22 wherein 5-HMF is used in combination with oleic acid, propionate, L-lysine and Nonivamide.

25. 5-HMF for use of claim 22 wherein 5-HMF is used in combination with oleic acid, propionate, L-lysine and capsaicin.

26. 5-HMF for use of claim 15 wherein 5-HMF is used in combination with Apigenin and/or hyperforin and/or PQQ and/or Urolithin A.

27. 5-HMF for use of claim 26 wherein 5-HMF is used in combination with Apigenin, hyperforin and PQQ.

28. 5-HMF for use of claim 26 wherein 5-HMF is used in combination with Apigenin, hyperforin and Urolithin A.

29. 5-HMF for use of claim 26 wherein 5-HMF is used in combination with Apigenin, hyperforin, PQQ and Urolithin A.

30. 5-HMF for use of claim 15 wherein 5-HMF is used in combination with sulphoraphane and/or allicin and/or PQQ and/or propionate and/or Urolithin A.

31. 5-HMF for use of claim 30 wherein 5-HMF is used in combination with sulphoraphane, allicin and propionate.

32. 5-HMF for use of claim 30 wherein 5-HMF is used in combination with sulphoraphane, PQQ and propionate.

33. 5-HMF for use of claim 30 wherein 5-HMF is used in combination with sulphoraphane, PQQ, propionate and Urolithin A.

34. 5-HMF for use of claim 30 wherein 5-HMF is used in combination with sulphoraphane, propionate and Urolithin A.

35. A composition comprising 5-HMF and at least one further active agent, preferably a further agent for modulation of mitochondrial function.

36. The composition of claim wherein the at least one agent is selected from the group consisting of one or more natural substances and one or more non-natural substances.

37. The composition of claim 36 wherein the one or more natural substances are selected from the group consisting of apigenin, quinine, PQQ, sulphoraphane, nonivamide, capsaicin, dehydrocapsiate, hyperforin allicin, DHC, ascorbic acid, spermidine, oleate, propionate, L-lysine, D-glucose, Utrolithin A, selenium, zinc, N-acylcysteine, Nacystelyn and N-acylcysteine amide.

38. The composition of claim 36 wherein the non-natural substance is selected from metformin, statins and thiazolidinones.

39. The composition of claim 38 wherein the statin is selected from the group consisting of one or more of atorvastatin, fluvastatin, lovastatin, pitavastatin, pravastatin, rosuvastatin calcium and simvastatin.

40. The composition of claim 38 wherein the thiazolidinone is selected from the group consisting of one or more of rosiglitazone, troglitazone, and pioglitazone.

41. The composition of claim 37 comprising 5-HMF in combination with ascorbic acid and/or Spermidine and/or Urolithin A.

42. The composition of claim 41 comprising 5-HMF in combination with ascorbic acid and Spermidine.

43. The composition of claim 41 comprising 5-HMF in combination with ascorbic acid, Spermidine and Urolithin A.

44. The composition of claim 37 comprising 5-HMF in combination with dehydrocapsiate and/or oleic acid and/or propionate and/or L-lysine and/or Nonivamide and/or capsaicin.

45. The composition of claim 44 comprising 5-HMF in combination with dehydrocapsiate, oleic acid, propionate and L-lysine.

46. The composition of claim 45 comprising 5-HMF in combination with oleic acid, propionate, L-lysine and Nonivamide.

47. The composition of claim 45 comprising 5-HMF in combination with oleic acid, propionate, L-lysine and capsaicin.

48. The composition of claim 37 comprising 5-HMF in combination with Apigenin and/or hyperforin and/or PQQ and/or Urolithin A.

49. The composition of claim 48 comprising 5-HMF in combination with Apigenin, hyperforin and PQQ.

50. The composition of claim 48 comprising 5-HMF in combination with Apigenin, hyperforin and Urolithin A.

51. The composition of claim 48 comprising 5-HMF in combination with Apigenin, hyperforin, PQQ and Urolithin A.

52. The composition of claim 37 comprising 5-HMF in combination with sulphoraphane and/or allicin and/or PQQ and/or propionate and/or Urolithin A.

53. The composition of claim 52 comprising 5-HMF in combination with sulphoraphane, allicin and propionate.

54. The composition of claim 52 comprising 5-HMF in combination with sulphoraphane, PQQ and propionate.

55. The composition of claim 52 comprising 5-HMF in combination with sulphoraphane, PQQ, propionate and Urolithin A.

56. The composition of claim 52 comprising 5-HMF in combination with sulphoraphane, propionate and Urolithin A.

57. The composition according to any one of claims 35 to 56 further comprising at least one pharmaceutically or nutraceutically acceptable carrier, excipient and/or diluent.
